(19) United States
(12) Patent Application Publication
(10) Pub. No.: US 2007/0088203 A1
(43) Pub. Date: Apr. 19, 2007

(54) Surgical assemblies and methods for visualizing and performing surgical procedures in reduced-access surgical sites

(76) Inventor: Liming Lau, Palo Alto, CA (US)

Correspondence Address: LAW OFFICE OF ALAN W. CANNON, 942 MESA OAK COURT, SUNNYVALE, CA, 94086, US

(21) Appl. No.: 11/503,463

(22) Filed: Aug. 10, 2006

Related U.S. Application Data
(63) Continuation of application No. 11/137,255, filed on May 25, 2005.

(51) Int. Cl. A61B 1/32

(52) U.S. Cl. 600/205

(57) ABSTRACT

Assemblies and methods for carrying out a procedure on an organ or tissue in a closed or restricted space surgical site. A low profile head is configured and dimensioned to be delivered to a target location on the organ or tissue through an opening in a patient and having a contact surface configured to contact a surface of the organ or tissue at the target location. An elongated member extends proximally from the low profile head and has sufficient length so that a proximal end portion of the elongated member extends out of the opening in the patient when the contact surface of the head contacts the organ or tissue at the target location. The elongated member has a lumen extending longitudinally therethrough. An endoscope is dimensioned to be received in the lumen and has an elongated shaft of sufficient length such that when a distal tip of the endoscope extends into the head, and the contact surface contacts the organ or tissue at the target location, a proximal end portion of the elongated shaft of the endoscope extends out of the opening in the patient.

### CROSS-REFERENCE

This application is a continuation-in-part application of co-pending U.S. application Ser. No. 11/137,255, filed May 25, 2005, which is incorporated herein by reference in its entirety and to which application we claim priority under 35 USC §120.

### FIELD OF THE INVENTION

The present invention pertains to the field of surgical instruments and techniques, and more particularly to surgical instruments and techniques for performance in reduced or restricted working spaces.

### BACKGROUND OF THE INVENTION

Certain surgical procedures require the surgeon to perform delicate surgical operations on tissues within the body that are moving or otherwise unstable. The ability to stabilize or immobilize a surgical site provides greatly improved surgical accuracy and precision and reduces the time required to complete a particular procedure. A large and growing number of surgeons are performing successful coronary artery bypass graft (CABG) surgery on the beating heart by temporarily stabilizing or immobilizing a localized area of the beating heart. Methods and apparatus for performing a CABG procedure on a beating heart are described in U.S. Pat. Nos. 5,894,843 and 5,727,569 to Benetti et al., the disclosures of which are herein incorporated by reference.

In a typical CABG procedure, a blocked or restricted section of coronary artery, which normally supplies blood to some portion of the heart, is bypassed using a source vessel or graft vessel to re-establish blood flow to the artery downstream of the blockage. This procedure requires the surgeon to create a fluid connection, or anastomosis, between the source or graft vessel and an arteriotomy or incision in the coronary artery. Forming an anastomosis between two vessels in this manner is a particularly delicate procedure requiring the precise placement of tiny sutures in the tissue surrounding the arteriotomy in the coronary artery and the source or graft vessel.

The rigors of creating a surgical anastomosis between a coronary artery and a source vessel or graft vessel demands that the target site for the anastomosis be substantially motionless. To this end, a number of devices have been developed which are directed to stabilizing a target site on the beating heart for the purpose of completing a cardiac surgical procedure, such as completing an anastomosis.

Stabilization may be provided using a device that provides a mechanical or compression force to the tissue or by a device which applies a negative pressure or suction to the tissue. Representative devices useful for stabilizing a beating heart are described, for example, in U.S. Pat. Nos. 5,894,843; 5,727,569; 5,836,311 and 5,865,730.

As beating heart procedures have evolved, regardless of whether compression or negative pressure has been used to stabilize or immobilize the heart, new challenges have arisen. For example, surgeons may gain access to the heart using a number of different approaches, both open and closed chest, such as through a sternotomy, mini-sternotomy, thoracotomy or mini-thoracotomy, or less invasively through a port provided within the chest cavity of the patient, e.g., between the ribs or in a subxyphoid area, with or without the visual assistance of a thoracoscope. Accordingly, the devices used to stabilize the heart must be configured to accommodate the particular approach chosen. For example, when a closed chest approach is used such as a port access approach wherein the device is introduced into the body cavity through a small access port or stab wound, the device must be designed to be advanced through such small openings without damaging the device or any internal body structures. A continuing need remains for new and better instruments that are capable of being delivered through small openings and still function satisfactorily in a closed-chest environment.

Furthermore, in addition to addressing delivery problems of instruments though small access openings, the working space within a closed-chest surgical environment is extremely limited, allowing much less room to maneuver the instruments, as compared to the space provided in an open-chest surgical site, once they have been successfully delivered or placed in the operative site. Thus, new and better approaches, tools and techniques for controlling instruments in a closed chest environment are needed.

Still further, during closed chest procedures it may not be possible for a surgeon to see a surgical target location, either directly (e.g., through an incision) or indirectly (e.g., via an endoscope introduced through an incision or port). For example, if the chest cavity is entered through a right lateral incision, a target area on the left lateral side of the heart may not be visible from the incision location. In such a situation, the surgeon must either place one or more devices or carry out one or more procedural steps at the surgical target area on the left or right lateral side of the heart “blindly” (i.e., without visualization of the activity/placement), or must create an additional incision through the left lateral chest to permit viewing, either directly or indirectly.

Blind placement of a device is clinically undesirable due to the risk of placing the device over a coronary artery, which could result in vessel injury, hemodynamic compromise, and/or obstruction of the target area. Making one or more additional incisions is also undesirable because of the increased potential for post-operative patient discomfort, increased risk of wound complications, etc, and because it is contrary to the minimally-invasive nature of a closed chest procedure, by making it more invasive.

As such, there is continued interest in the development of new devices and methods for further minimizing the invasiveness of surgical procedures, to provide devices and techniques for improved visualization with less invasive requirements, such that visualization can be carried out through an existing opening through which a device is passed to perform a surgical task, thereby mitigating the risks of blind placement and additional incisions or ports of entry that are currently required. Of particular interest would be the development of such devices and methods of use which may be used for minimally invasive procedures, such as endoscopic or port access procedures (e.g., between the ribs or in a subxyphoid area).

### SUMMARY OF THE INVENTION

An assembly for carrying out a procedure on an organ or tissue in a closed or restricted space surgical site is provided, including a low profile head configured and dimensioned to be delivered to a target location on the organ or tissue through an opening in a patient and having a contact surface configured to contact a surface of the organ or tissue at the target location; and an elongated member extending proximally from the low profile head and having sufficient length so that a proximal end portion of the elongated member extends out of the opening in the patient when the contact surface of the head contacts the organ or tissue at the target location. The elongated member has a lumen extending longitudinally therethrough. An endoscope is dimensioned to be received in the lumen and has an elongated shaft of sufficient length such that when a distal tip of the endoscope extends into the head, and the contact surface contacts the organ or tissue at the target location, a proximal end portion of the elongated shaft extends out of the opening in the patient.

An organ manipulator assembly for manipulating an organ in a closed-cavity or reduced access surgical site is provided, including: a suction head having an interior space surrounded by a contact member configured to seal with a surface of the organ when the contact member is contacted to the surface and negative pressure is applied to the interior space; and an elongated member extending proximally from the suction head and having sufficient length so that a proximal end portion of the elongated member extends out of an opening in a patient through which the suction head is inserted and advanced to contact the organ at a target location. A lumen extends longitudinally through the elongated member. An opening through the suction head connects the lumen with the interior space. An endoscope is dimensioned to be received in the lumen, while still allowing vacuum flow through the lumen, and is insertable through the lumen so that a distal tip of the endoscope extends into the interior space.

A method of carrying out a procedure on an organ or tissue in a closed or restricted space surgical site includes: providing an assembly having a low profile head and an elongated member with an endoscope received within a lumen of the elongated member such that a distal tip of the endoscope is positioned to visualize placement of the low profile head; inserting the low profile head through an opening in a patient; advancing the low profile head to a region of the organ or tissue that includes a target location on which a procedure is to be performed; visualizing a location of the head, relative to the target location, through the endoscope; and performing the procedure at the target location with the head after visually confirming that placement of the head, relative to the target location, is acceptable.

These and other features of the invention will become apparent to those persons skilled in the art upon reading the details of the devices and methods as more fully described below.

### BRIEF DESCRIPTION OF THE DRAWINGS

FIG. 1 shows a manipulator assembly in a disassembled configuration.

FIG. 2 shows an enlarged view of the manipulator head portion of the assembly of FIG. 1.

FIG. 3 illustrates a malleable stylet.

FIG. 4 illustrates a cross-sectional view of a flexible shaft of a fiber-optic type of endoscope where a malleable stylet has been built into the flexible shaft.

FIG. 5A shows a partial cutaway view of the assembly of FIG. 1 in an assembled configuration.

FIG. 5B illustrates an angled distal tip of an endoscope.

FIG. 6 illustrates one embodiment of use of the assembly of FIG. 1.

FIG. 7A shows another example of an organ manipulator for use in closed-chest or limited space surgical sites according to the present invention.

FIG. 7B illustrates a distal end portion of a grasper.

FIG. 7C is a side view of a suction member that may be employed in a manipulator assembly according to the present invention.

FIG. 7D is a partial, bottom view of the manipulator assembly of FIG. 7A.

FIG. 7E shows a manipulator assembly in which grab members comprise nubs extending from locations distributed over the superior surface of a suction member.

FIG. 7F is a side view of a suction member showing a variation of grab members.

FIG. 7G is a partial top view of a manipulator assembly showing another variation of grab member.

FIG. 7H shows another example of a manipulator assembly provided with grab members.

FIG. 7I shows still another example of a manipulator assembly provided with grab members.

FIG. 8A is a partially cutaway view of an example of a tubular elongated member that may be employed in the manipulator assembly of FIG. 1.

FIG. 8B is a partially cutaway view of another example of a tubular elongated member that may be employed in the manipulator assembly of FIG. 1.

FIG. 8C shows an assembly formed by bending a malleable manipulator assembly, in use performing manipulation of an organ.

FIG. 9 shows a stabilizer assembly in a disassembled configuration.

FIG. 10 shows an alternative stabilizer device to the stabilizer device shown in FIG. 9.

FIG. 11 illustrates an alternative arrangement for placement of the distal tip of the endoscope of the assembly of FIG. 9.

FIG. 12 illustrates an alternative arrangement for placement of a distal tip of an endoscope with regard to the stabilizer device of FIG. 10.

FIG. 13 illustrates one embodiment of use of the assembly of FIG. 9.

### DETAILED DESCRIPTION OF THE INVENTION

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a shaft” includes a plurality of such shafts and reference to “the head” includes reference to one or more heads and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The term “open-chest procedure” refers to a surgical procedure wherein access for performing the procedure is provided by a full sternotomy, wherein the sternum is incised and the patient's ribs are separated using a sternal retractor to open the chest cavity for access thereto.

The term “closed-chest procedure” refers to a surgical procedure wherein access for performing the procedure is provided by one or more openings which are much smaller than the opening provided by an open-chest procedure, and wherein a traditional sternotomy is not performed. Closed-chest procedures may include those where access is provided by any of a number of different approaches, including mini-sternotomy, thoracotomy or mini-thoracotomy, or less invasively through a port provided within the chest cavity of the patient, e.g., between the ribs or in a subxyphoid area, with or without the visual assistance of a thoracoscope.

As alluded to above, surgical procedures carried out on a patient by “closed-chest” procedures allow much less free space for the surgeon to work in than do more conventional “open-chest” procedures such as those where access is gained through a sternotomy, for example. As such, the instruments used during closed-chest procedures must be as non-obstructive as possible and require only minimal amounts of space for their use.

Devices and Methods

The present invention provides devices and methods that are useful for performing surgical procedures where working space is limited, and provide visualization capabilities for target areas that are typically not visible from an access incision through which the devices are inserted.

FIG. 1 shows a manipulator assembly 1 in a disassembled configuration. While usable under other surgical conditions, assembly 1 is particularly well-suited for use in closed-chest or limited space surgical sites, and provides visualization capability for visualizing sites that would otherwise not be visible under previous practice techniques. Organ manipulator 10 includes a low profile head 12 configured for use in closed-chest or limited space surgical sites, wherein the low profile facilitates insertion of head 12 and occupies only a very narrow space between an organ to be manipulated and tissues surrounding the organ. Head 12 may be in the form of a low profile suction member as a single suction member (e.g., suction cup) as shown in FIG. 1, configured to engage with and maintain a grasp of an organ, such as a heart for example, with a sufficient strength so as not to break contact when suction member 12 is moved, so that the organ is moved by moving suction member 12. Suction member 12 may be made of silicone or other biocompatible elastomer sufficient to maintain the suction between suction member 12 and the organ as the organ is moved via movement of suction member 12. Further alternatively, head 12 may be made up of more than one suction cup, or other multiple members adapted to apply negative pressure, such as multiple arms having ports, or of a single contact member configured with a space between a top of the member and a contact surface of the suction member to generate a negative pressure in the space when the contact member seals with the surface of an organ, for example. Further, suction member 12 may be provided with a plurality of suction ports to deliver negative pressure to the interior thereof. Further design variations that may be applied to suction member 12 are described in co-pending, commonly assigned U.S. Patent Application Publication No. 2005/0010197, U.S. Patent Application Publication No. 2003/0009080, and in U.S. Pat. No. 6,338,712. U.S. Patent Application Publication Nos. 2005/0010197, 2003/0009080, and U.S. Pat. No. 6,338,712 are each hereby incorporated herein, in their entireties, by reference thereto.

Manipulator 10 further includes an elongated member 13 including a lumen that functions as a suction conduit as well as receiving an endoscope therethrough as will be described below. Elongated member 13 extends from head 12 to a proximal hub 14 that is configured to be fluidly connected with a source of vacuum, for delivery of negative pressure to head 12 via elongated member 13, with the suction source being located outside of the body. Thus, elongated member 13 fluidly connects head 12 with hub 14. Elongated member 13 may be a flexible, air impermeable tubing or alternatively may further employ a structure for facilitating manipulation of head 12, such as a torque tube which is flexible in bending, but resistant to torquing motions.

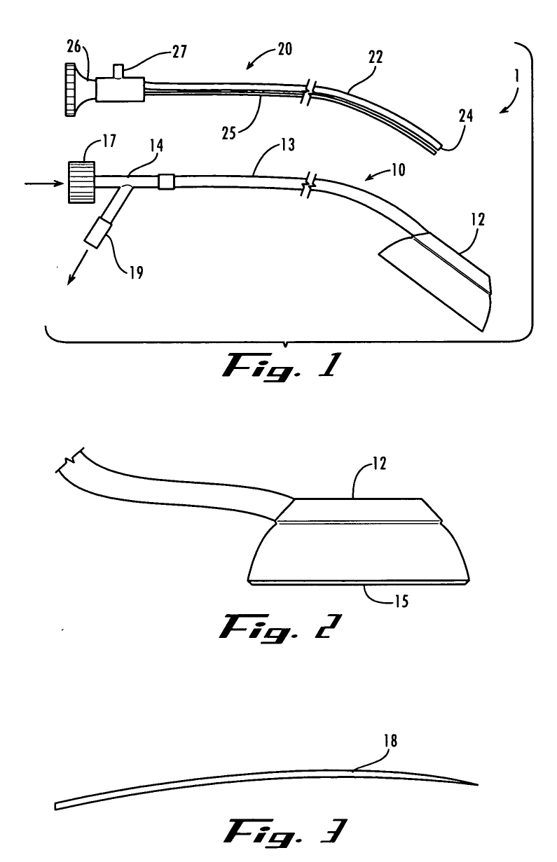

Further, each suction member 12 may be provided with seal 15, as shown in FIG. 2, which may be made of closed-cell foam that enhances the ability of suction member 12 to conform to the surface of the organ and to establish a vacuum seal therewith. In one embodiment, manipulator 10 includes a flexible suction member 12 with a substantially circular opening having an inside diameter of about one to about one and a half inches and an outside diameter of about one and a half to about two inches, with seal 15 extending from the bottom surface of the flexible member 12 by a thickness of about ⅛to ⅜inches. Elongated member 13 may extend from suction member 12 by about twelve to eighteen inches (although this length may be increased or decreased depending upon the surgical application that the manipulator is designed to be used for), and comprises a torque tube that is flexible in bending but resistant to torquing motions about the longitudinal axis of elongated member 13. It should be noted here that these dimensions are with regard to a particular embodiment, and may vary depending upon the patient, size of the organ to be manipulated, and location of entry though which the manipulator 10 is inserted, etc.

Assembly 1 further includes an endoscope 20 configured to be received within the lumen 16 of elongated member 13. Although endoscope 20 could have a rigid elongated shaft, this reduces the ability to maneuver the assembly in closed-cavity surgical sites and reduces the capability to view surgical target locations that are difficult to access and view. Accordingly, endoscope 20 typically includes a flexible shaft 22 and distal tip 24. Distal tip 24 may be an optical tip that delivers light via optical fibers, optionally through one or more lenses and receives reflected light from the surgical site, the reflected light optionally passing through one or more lenses, and being returned to the proximal end of endoscope 20 via optical fibers. Alternatively, tip 24 may be provided with one or more charge couple devices (CCD's) for receiving the light reflected from the surgical site, converting the reflected light received to electrical signals, and passing image information proximally through shaft 22 along one or more wires in the form of electrical signals. There may be a lens (objective) provided at or in distal tip 24 for image management/enhancement (e.g., to narrow or widen the included angle of the field of view and/or the amount of tilt of the field of view). Typically, when an objective is provided, it is the distal most component in the distal tip, to control (bend) the incoming light before it reaches the optical fibers or the CCD sensor. An image of the surgical site is formed at ocular 26 by the light received through fiber optics (in the case of a fiberoptic endoscope). For an endoscope employing a CCD system, an ocular is typically not provided at the proximal hub of the endoscope. Rather, a connector-type port is provided at the proximal hub for an electrical cable from a camera module to be connected thereto, and such arrangements are currently available in the market. An example of this type of electrical connector arrangement is shown in the Schoelly Flexiscope® product, for example see http://www.schoelly.de/english/industrial/flexiscope/index.html#. Ocular 26 may be provided at a proximal end portion (proximal hub) of the shaft 20 as shown in FIG. 1. An electrical connection from an endoscope employing a CCD system is also typically located on the proximal hub of the endoscope, as noted. Additionally, or alternatively, the proximal end portion of endoscope 20 may be connected to a camera adapter which attaches to the occluder 26. The port 27 on the side of the proximal hub is a traditional light cable attachment port that may be an optical or electrical feed from the endoscope to a camera module, so that photos can be captured and/or live visualization of the surgical site can be provided on a monitor. Images may be provided to the proximal end of the endoscope via fiber optics or electrical wires (for a CCD tipped scope), under known current practices. The port 27 on the side of the proximal hub is a traditional light cable attachment port. These connections may be integrated and streamlined to into the proximal end of the endoscope, e.g., called a “probe” in the Schoelly Flexiscope® product, http://www.schoelly.de/english/industrial/flexiscope/index.html#.

Distal tip 24 of endoscope 20 may be angled with respect to a normal to the longitudinal axis of shaft 22 or configured to provide a field of view that is angled with respect to the normal to the longitudinal axis of shaft 22, to provide a downward looking field of view 33 as illustrated in FIG. 5A. Light rays emitted from distal tip 24 may be angled by a prism objective, or by beveling the distal ends of the light input fibers. Rays entering back into the endoscope (after being reflected from a viewing target) are angled as they pass through the optional prism objective, prior to being transmitted by optical fibers to the proximal end of the endoscope. Alternatively, for a CCD-tipped endoscope, the CCD chip may be mounted at an angle to face the desired field of view, without the use of an objective lens or prism. The angled field of view 33 provided by this configuration allows distal tip 24 to be introduced into head 12 from the side of the head, thereby maintaining the low profile of the assembly. Otherwise, tip 24 would need to be introduced through the top of head 12 and this would increase the height (profile) of the head and require more space during use, particularly since optical fibers withstand tight bends. FIG. 5B illustrates a distal end portion of shaft 22 and distal tip 24 showing the angling α of distal tip 24 with respect to normal 23′ to the longitudinal axis 23 of shaft 22. For example, tip 24 may be angled by an angle α of about 30 to about 45 degrees to provide the desired field of view 33 with respect to head 12 as described below. The angling may be accomplished, for a fiberoptic type endoscope by beveling the distal end of the optical fibers at the angle α Alternatively, or also for other types of endoscopes (including CCD versions, as described above), angling of light emitted from distal tip 24 and entering distal tip 24, with respect to the normal 23′ may be accomplished by placing a prism at the distal tip 24, to divert the light by the desired angle α.

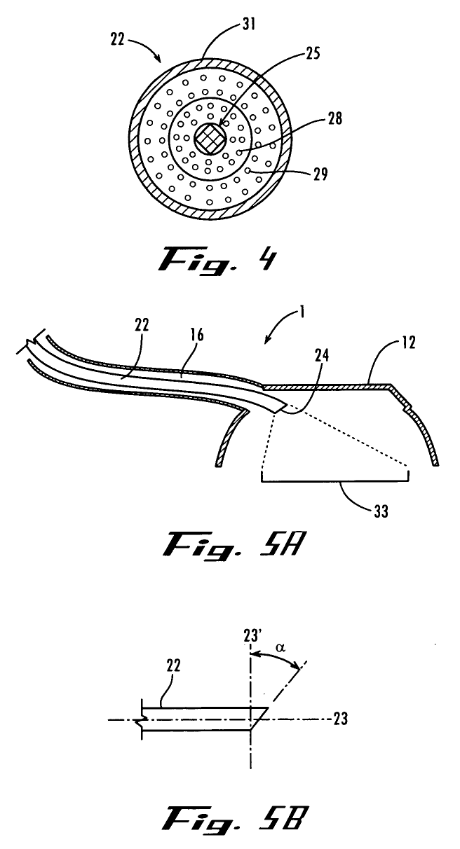

As noted, endoscope shaft 22 may be flexible to allow the endoscope to be bent, flexed and shaped to optimize ease of insertion, both into device 10 as well as into a closed surgical site. Additionally, endoscope 20 may be provided with a malleable stylet 25 that runs coaxially along all or a portion of flexible shaft 22, or that may be built into the flexible shaft 22 (FIG. 4). When built in, and when a prism or other deflecting lens is placed at the distal end of shaft 22 (distal tip 24), malleable stylet ends proximally of the location of the prism/lens. Malleable stylet 25 allows flexible shaft 22 to be flexed or bent, and then retains the flexible shaft in that configuration until such time as an operator decides to further bend or flex, or re-bend or shape the shafts 22 and stylet 25 into another configuration. Malleable stylet 25 may be made from a malleable metal, such as annealed stainless steel, nickel-titanium alloy or malleable polymer, for example. Malleable stylet 25 may include sufficient stiffness or rigidity, so that forces imposed on it during insertion into device 10, as well as forces imposed on it during delivery to the closed surgical space are not sufficient to bend or deform the shaft 25. However, shaft 25 is sufficiently flexible so that it can be readily bent and reshaped manually by a user of the device.

Alternatively, or additionally, a malleable stylet 18 (FIG. 3) or other shaping member may be inserted through elongated member 13 to maintain elongated member 13 in a desired shape. Stylet 18 may be removable from elongated member, or may be integral therewith. In either case, stylet 18 runs coaxially with shaft 22 (and optionally, shaft 25) when endoscope 20 is inserted into elongated member 13. In practice, shaft 25 may facilitate the insertion of the endoscope 20 by providing more rigidity to the flexible shaft 22 and therefore the arrangement shown in FIG. 1 may provide for easier insertion of endoscope 20 into device 10.

FIG. 4 illustrates a cross-sectional view of a flexible shaft 22 of a fiber-optic type of endoscope 20 in which malleable stylet 25 has been built into the flexible shaft. In this embodiment, malleable stylet 25 is disposed centrally within shaft 22, surrounded by a plurality of image output optical fibers 28, which in turn are surrounded by a plurality of light input optical fibers 29. The interstitial spaces between the fibers 28,29 may be filled using an elastomeric material, such as silicone, silicone elastomer, etc., or may be left unfilled. The use of filler may be advantageous in helping to prevent fluid ingress, including during cleaning and sterilization of the endoscope. In practice, light is delivered from a light source (which may be external to the endoscope 20 and connected to light input optical fibers) through light input optical fibers 29 and emitted from tip 24. The emitted light is reflected from the object to be viewed (e.g., organ surface and portion of head 12) to image output optical fibers 29 which deliver the reflected light to ocular 26 and/or camera adapter 26. A flexible outer cladding 31 surrounds the internal components of the flexible shaft to maintain the integrity of the internal components, as well as seal them off from blood or other contaminants. Outer cladding 31 may typically be made from an elastomer, such as silicone, other tubing materials approved for use in catheters, biocompatible electrical cable coating materials, or other biocompatible materials that are flexible, and, for a reusable endoscope, able to withstand reuse and reprocessing such as heat-, radiation- and/or chemical-based sterilization processes.

Proximal hub 14 of device 10 (see FIG. 1) facilitates dual entry of both suction and the endoscope into device 10. Endoscope 20 is insertable through hemostatic valve coupling 17 so that endoscope 20 may be advanced through lumen 16 until tip 24 is positioned for visualization of the placement of head 12. A suction port 19 is provided in proximal hub 14 that is connectable to an external source of suction. The hemostatic valve 17 is sufficient to maintain the suction in lumen 16 (e.g., see FIG. 3) when a suction source has been coupled to suction port 19, so that endoscope 20 may remain in place within the lumen and does not need to be removed. Further, valve 17 permits endoscope 20 to be removed from the port thereof and from device 10 while continuously applying vacuum through port 19 and lumen 16 without significant loss of vacuum pressure. Thus vacuum may be applied to suction member 12 by connecting vacuum port 19 with a source of suction, and vacuum may be maintained simultaneously with the use of (including insertion and/withdrawal of) endoscope 20. Whichever configuration is used, e.g., flexible shaft 22 and coaxial malleable stylet 25, flexible shaft 22 having an integrated malleable stylet, flexible shaft 22 with malleable stylet 18, etc., the combined cross sectional areas of any shafts and stylets having been inserted into lumen 16 are less than the cross sectional area of the lumen, to allow suction to be delivered in the remaining space in the lumen.

FIG. 5A is a partial sectional illustration of an endoscope 20 having been inserted into a low profile manipulator 10, as described. In FIG. 5A, flexible endoscope shaft 22 incorporates a malleable stylet 25 and has a cross sectional area less than the cross sectional area of lumen 16, so that suction is communicable via the free space of lumen 16 shown, even when endoscope 20 is fully inserted into the device 10. Note in FIG. 5A that distal tip 24 is positioned within the interior space of head 12 and is angled downward to provide an appropriate view of the positioning of head 12 relative to the organ. The field of view of tip 24 in the position shown is indicated by 33. Endoscope 20 can be further manipulated, such as by rotating shaft 22 or inserting tip 24 further into the interior space of head 12 or withdrawing tip 24 closer to the inner wall of head 12, to alter the field of view. All of these manipulations can be performed outside of the patient, at a proximal end portion of endoscope 20 located proximally of the proximal hub 14.

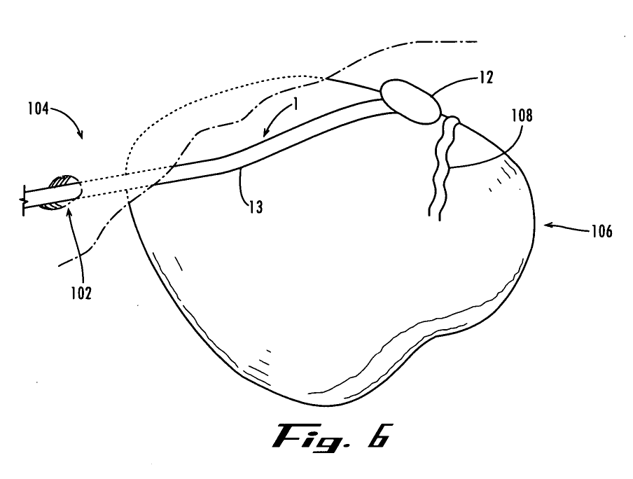

FIG. 6 shows a cutaway view 100 showing use of a manipulation assembly 1 in the performance of manipulation or positioning of the heart during a closed-chest, beating heart, coronary artery bypass graft (CABG) procedure. In this procedure, a small opening, such as a small thoracotomy 102, port or other small incision has been formed in the patient's chest 104, such as in the third or fourth intercostal space, for example, of a size large enough to pass head 12 (in a compact configuration) therethrough, and to allow sufficient space for the surgeon to work through in performance of dissection and anastomosis procedures. Thus, when performing a procedure through a thoracotomy, the surgeon may be able to work through this same thoracotomy to perform the surgical procedure. When the endoscope is to be inserted through a port, at least one additional port is typically required to insert working instruments through to perform the surgical procedure. However, a primary opening other than a thoracotomy may alternatively be employed, such as a mini-sternotomy, sub-sternal opening, or the like.

In use, suction member 12 may be folded into a cylindrical or taco shape and passed through an opening (e.g., opening 102) to be placed on the surface of an organ to be manipulated. Passing the suction member 12 through an opening may be performed either prior to insertion of the endoscope, or after the endoscope has already been inserted. In the view of FIG. 6, assembly 1 has been inserted through opening 102 on the left lateral side of the patient, and head 12 has been advanced to a site on the right lateral side of the heart 106. Endoscope 20 provided within manipulator 10 may be used to view the position of head 12 relative to the heart 106 to ensure that head 12 does not rest on a coronary artery 108 prior to applying negative pressure to head 12 to attach to the surface of the heart. The partial rigidity of elongated member 13 provided by the one or more malleable stylets within the elongated member facilitate both distal movements of the assembly, as well as lateral movements for repositioning head 12 to a desired location. Proximal movements may be accomplished by simply pulling on the assembly at the proximal end portion of elongated member 13 that extends out of the patient.

Once a target (desired) location for attachment to the heart has been accessed and visual confirmation has been obtained that head 12 is acceptably positioned, suction (negative pressure is applied though lumen 16 via suction port 19 which causes the contact surface of head 12 to seal with the surface of the heart as a negative pressure is generated within the internal cavity of the head 12, and head 12 attaches to the surface of the heart with sufficient force such that movement of head 12 causes movement/repositioning of the heart without breaking the seal between head 12 and the surface of the heart 106.

If desired, the malleable stylet 25 or 18 may be shaped prior to insertion of the manipulator 10 into the body or prior to insertion of the endoscope 20 into the manipulator 10, to facilitate access to the targeted location on the organ. Further, if the initial shaping of the malleable stylet 25 or 18 is not acceptable and does not facilitate the placement of the assembly as well as expected, the endoscope and/or assembly can be withdrawn to reshape the malleable stylet 25 or 18 for a better approximation of shape to the pathway to be traversed upon installing the assembly 1.

In closed chest procedures (or in closed-cavity procedures, such as where the assembly is being used to manipulate an organ in the abdominal cavity, for example), it may not be possible for the surgeon to see the target location either under direct vision (e.g., by “eyeballing” the location through a direct line of sight through the incision) or via a standard endoscope inserted through the incision, such as a rigid shaft endoscope. For example, in FIG. 1, the target area that the head 12 overlies is not in a direct line of sight with opening 102, as the heart is blocking the direct line of vision. Also, insertion of a rigid endoscope into opening 102 would not provide visualization of the target area, as the viewing tip of the endoscope would not be able to be directed around the curvature of the heart surface to align it with the target area. Although another incision could be placed in the left lateral chest of the patient, this is not desirable, for reasons indicated earlier. The flexible endoscope 20 resident within the manipulator 10 not only provides visualization of the target area, but does so in a manner that does not take up any additional space within the close confines of the closed cavity surgical site. That is, neither an additional shaft is required to be inserted through opening 102, nor is there an additional instrument taking up space adjacent head 12.

Once head 12 has been attached to the surface of the heart 106 in the example described above, the heart may be repositioned by pushing or pulling on elongated member 13 from a location outside of the patient. Side to side movements may also be accomplished in a manner as described above. Continue visual monitoring may be performed at any time or all during these procedures, to ensure that head 12 remains attached to the surface of the heart. When manipulation of the heart is no longer required, the vacuum can be discontinued, thereby releasing the grip of the head on the surface of the heart and then assembly 1 can be withdrawn from the patient.

Although the assemblies described thus far each have a removable endoscope 20, it is noted that any of the embodiments described may have endoscope 20 provided integrally in device 10. Endoscope 20, in such arrangement may still be provided with limited relative axial movement with regard to device 10 to allow varying the field of view in the proximal-distal directions. Further in any of the removable or non-removable (integral) arrangements head 12 may be provided to be removable and replaceable, so that a new head may be provided for each procedure.

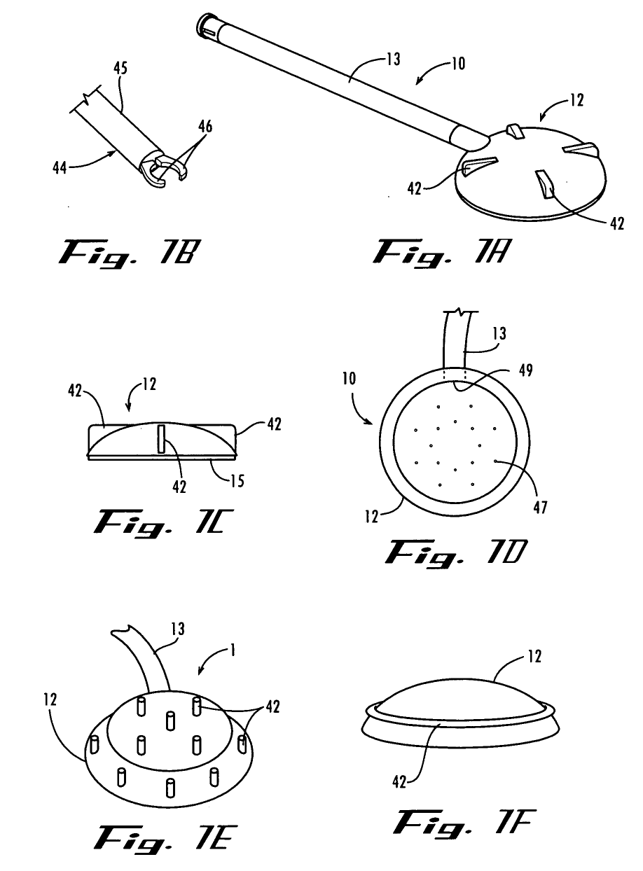

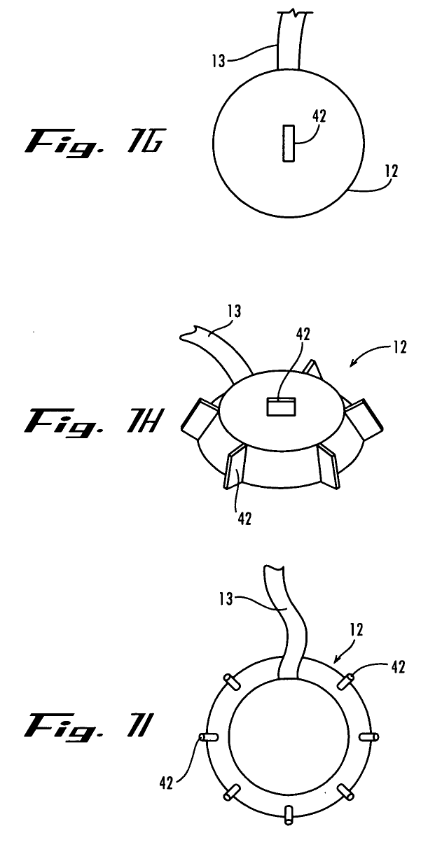

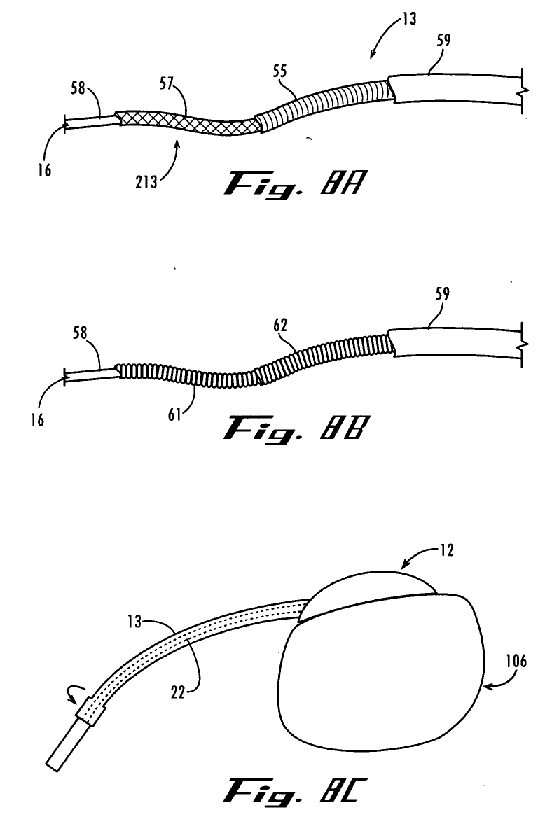

In some situations, it may be desirable to use an additional tool to aid in positioning head 12 prior to attaching it to the target surface of the organ to be manipulated. In such situations, head 12 may be provided with one or more low profile grab members 42 around the periphery or spaced about the superior surface thereof, as shown in FIG. 7A, that extend from the superior surface of suction member 12 so that they can be readily grasped by a tool such as grasper 44 (see FIG. 7B). A typical grasper 44 includes a small diameter shaft 45 (e.g. in the vicinity of 5 mm outside diameter) which extends the jaws 46 of the grasper through an opening (e.g., opening 102, or other opening), while permitting an operator to operate the jaws from outside the patient to grab and release grab members 42 in the process of effectively moving and/or repositioning the device from which grab members 62 extend. Grasper 44 may be a conventional surgical tool and operated by a simple scissor-type actuator, for example, or other conventional, readily available grasping tool dimensioned to be useable through thoracotomy 102 or other opening or port through the chest wall. As shown, grab members 42 are configured as semi-circular flaps or fins that are integrally molded with suction member 12 to extend therefrom. However, grab members 42 may have other configurations, such as loops or other extensions extending from head 12 that may be easily grasped by grasper 44. Grab members 42 may be are typically grasped to position organ manipulator 10 on an organ in a desired position and orientation prior to applying suction, buy may also be used to move organ manipulator 10 and the organ after suction has been applied and organ manipulator 10 has become fixed to the organ. When using multiple graspers, one of the grab members may be is grasped by a second grasping tool before releasing a grasp of the same or another grab member by a first grasping tool. Alternatively, grab members may be simultaneously grasped by at least first and second grasping tools to triangulate forces on the head 12 to move it during positioning or manipulation. Such first and second grasping members may simultaneously engage the same grab member, or, more typically, different grab members.

FIG. 7C is a side view of the head 12 shown in FIG. 7A, illustrating the low profile nature of the grab members 42. It can be observed that the grab members 42 do not extend vertically above the highest point (in this case, the center) of the suction member in FIG. 7C.

Suction member 12 may also be provided with a layer of open-cell foam 47 or mesh, as shown in FIG. 7D, to act as a diffuser and prevent or substantially reduce the possibility of the surface of the organ from being sucked against one or more ports that establish the vacuum seal. Mesh or foam 47 does not cover the opening 49 through which the distal end of endoscope 20 is inserted, to prevent obscuring the view by the distal tip 24. Although shown with only one port 49 entering from the periphery of suction member 202, suction member 12 may be alternatively provided with a plurality of suction ports 49 to deliver negative pressure to the interior thereof. Further design variations that may be applied to suction member 12 are described in U.S. Publication No. 2005/0010197, published on Jan. 13, 2005, and titled “Organ Manipulator Apparatus”, in U.S. Patent Application Publication No. 2003/0009080, and in U.S. Pat. No. 6,338,712. U.S. Patent Application Publication Nos. 2005/0010197 and 2003/0009080, and U.S. Pat. No. 6,338,712 are each hereby incorporated herein, in their entireties, by reference thereto.

FIG. 7E is a partial view of a manipulator assembly 1 in which grab members 42 are formed as extending nubs and extend from locations distributed over the superior surface of head 12, i.e., are not limited to distribution around the perimeter of the upper surface. FIG. 7F shows another variation of grab members 42 in which a continuous, ring-like projection 42 is formed around head 12 to extend therefrom. FIG. 7G shows still another variation in which a single tab-like grab member 42 extends from the center or top of the superior surface of head 12. The example in FIG. 7H includes head 12 having a tab or flap 42 on the superior surface thereof, as well as rectangular shaped flaps 42 extending radially from the periphery of head 12. FIG. 71 shows another arrangement in which nubs 42 extend radially from the periphery of head 12. Grab members 42 may be grabbed and pulled, pushed and/or rotated to effect a desired positioning of head 12 on the surface of an organ.

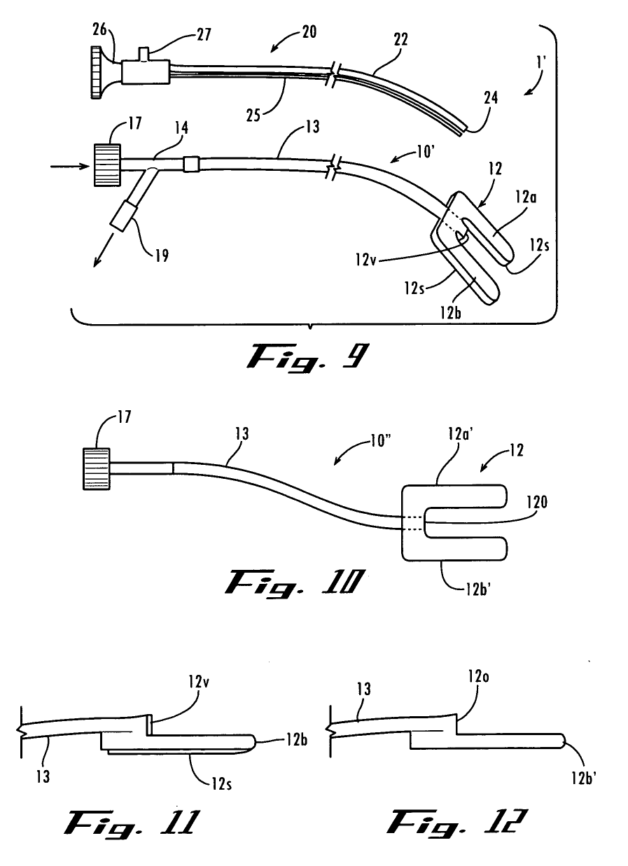

As noted earlier, elongated member 13 may be very flexible in bending but very stiff with regard to torsion. This may be made possible by overlaying a coil 55 (preferably a metallic coil, e.g., stainless steel or the like) over a braided tube 57 (see partially cutaway view of FIG. 8A) which is also preferably made of metal, such as stainless steel or the like. Tubular braid 57 may be formed over an inner polymeric tubular sleeve 58, which defines lumen 16. Braid 57 is typically encapsulated in sleeve 58, such as by molding, extrusion, forming, or the like. Sleeve 58 may be made from PEBAX® resins (polyether-block co-polyamide polymers), nylon, silicone, urethane or other flexible, biocompatible polymer, or combinations thereof, for example.

An outer polymeric sleeve 59 may be provided over coil 55, as also shown in FIG. 8A. Similarly, coil 55 may be encapsulated in sleeve 59, such as by molding, forming, extrusion or the like. Sleeve 59 may be made from any of the polymers described with regard to sleeve 58. The polymeric sleeves function to maintain vacuum integrity, i.e., so that no significant leakage of vacuum occurs along the sleeves 58, 59. Additionally, by embedding the coil and braid components into the respective sleeves, the sleeves add significant mechanical support to the coil and braid. This configuration provides a tubular structure that is very flexible with regarding to bending (e.g., with respect to it's longitudinal axis), but very stiff in torsion, so that torquing of elongated member 13 by an operator will transfer the torsional forces effectively to head 12 to effect repositioning or manipulation/movement of an organ attached thereto.

Alternatively, braid 57 may be slid over sleeve 58 and/or sleeve 59 may be slid over coil 55 or heat shrunk thereon, but if the coil and braid are not embedded in the sleeves, then the mechanical support by the sleeves decreases substantially.

FIG. 8B shows another configuration of elongated member 13 that is also very flexible in bending but stiff under torsion. In this example, oppositely wound spring coils 61,62 (preferably metallic, such as stainless steel of the like) are provided in the shaft. For example, a counterclockwise inner spring 61 may be provided over inner polymeric tubular sleeve 58, and a clockwise outer spring 62 may be provided over inner spring 61. Like the previous example, an outer sleeve 59 is provided over outer spring 62. Of course, this arrangement may be altered so that inner spring 61 is clockwise wound and outer spring 62 is counterclockwise wound. Spring 61 is typically encapsulated into sleeve 58 and spring 62 is typically encapsulated into sleeve 59, such as by any of the techniques described above with regard to the previous example. However the springs and coils may alternatively be slid into place over one another, although some mechanical support by the sleeves in such a configuration may be diminished.

In another embodiment, elongated member 13 may be a single unitary flexible tube, such as a polymeric tube defining lumen 16. In still another arrangement, elongated member may include a single flexible tube, such as a polymeric tube, with a malleable stylet 18 incorporated therein and running coaxially with lumen 16 of elongated member 13. In still another embodiment, elongated member may be a flexible tube having primary lumen 16, with a secondary lumen of smaller dimension running coaxially therewith, the secondary lumen being dimensioned to slidably receive malleable stylet.

The aforementioned configurations are particularly useful for endoscopically delivering and positioning device 10 or assembly 1 and subsequently performing a procedure at a target location on an organ (e.g., such as manipulation, although similar elongate members may be provided with different types of device heads for performing other procedures, at least one of which is described below). Placement of head 12, as well as endoscope 20 may be improved with the use of one or more malleable stylets, as described above.

By bending the stylet 25 of endoscope 20 prior to insertion of endoscope 20 into device 10, or by bending stylet 18 as desired and inserting it into elongated member, or by bending elongated member 13 as desired if malleable stylet 18 is integrated therewith, or by bending elongated member 13 as desired if endoscope 20 and thus stylet 25 are integrated therewith, an optimal or near optimal orientation angle of approach of the inferior surface of head 12 may be established with respect to the target location of the organ to be addressed by head 12, as illustrated in FIG. 8C. Further, once the organ 106 has been contacted and attached by head 12, as shown in FIG. 8C, rotation of torsionally stiff elongated member 13 transfers the rotational force to head 12 to turn it and the organ with it.

One method of using assembly 1 to perform manipulation/positioning of a heart during a closed chest procedure is now described. Endoscope 20 is inserted into elongated member 13 of device 10, through hemostatic valve 17 and advanced until distal tip 24 is in position to provide a field of view to allow the user a view of where head 12 is being placed. For example, endoscope 20 may be advanced to the position shown in FIG. 5A. This positioning may be directly visually confirmed by the user/surgeon, by flipping over head 12 to see the relative positioning of tip 24 with respect to head 12. The endoscope 20 is connected to a light source (and, optionally to a camera and/or monitor) and device 10 is connected to a suction source, although suction is not yet applied to the device.

Assembly 1 is next inserted though a port, small thoracotomy, or other small incision in a patient and is advanced toward the target surgical site. Head 12 may be compacted, such as by folding, rolling or otherwise compressing it for easier insertion through the incision in the patient. After passing through the incision, head 12 then elastically returns to its undeformed shape. Alternatively, graspers 44 or other surgical tool may be used to facilitate re-expanding heads to its undeformed/uncompressed state after passing through the incision. Light may be supplied through endoscope, even prior to passing head 12 through the incision. Once in its undeformed, operative configuration, head 12 will not obstruct visualization through tip 24 of endoscope 20 and so the advancement of the assembly 1 may be visualized to assist in directing the assembly to the target location.

Once in the target location, visualization of the field of view 33 over the area that head 12 is currently placed in can confirm whether or not the location is a desirable one for placement of head 12. In the case of a manipulator 10 with suction member 12, the operator/surgeon confirms that the suction member 12 does not overlie or intersect any coronary arteries. Upon such confirmation, suction is applied through suction port 19 and head 12 forms a seal with the surface of the heart, thereby engaging it under negative pressure. Subsequent pulling, twisting and/or pushing on elongated member, from a location outside of the body, transfers these forces to move head 12 and the heart attached thereto. After movement to a desired location or orientation, elongated member 13 may be fixed at its proximal end portion outside of the patient to maintain the current orientation of the heart to enable a further surgical procedure to be performed on the heart in its current orientation. For example, a clamp, or other mechanical fixation means may be applied to the proximal end portion of elongated member 13 to fix it relative to a stationary object outside the patient to prevent axial, as well as rotational movements thereof. Upon completion of the procedure, elongated member is released from fixation, and moved to replace the heart to its normal orientation. Vacuum is then released from device 10 and head 12 releases its engagement with the surface of the heart. Assembly 1 can then be withdrawn from the patient. Application of light through tip 24 may be maintained if desired, even during removal of the assembly, to provide visual feedback to the surgeon of the removal process of assembly, and to ensure that the removal process proceeds safely and without incident.

Alternative methods to that described may be practiced with assembly 1. For example, head 12 may be inserted through the incision prior to insertion of endoscope 20 into device 10. In such case, endoscope 20 may be inserted immediately after passing head 12 through the incision and ensuring that head 12 is in its operable (noncompressed) configuration. Alternatively, head 12 may be advanced part way along the route to the target surgical site prior to inserting endoscope 20. In either case, endoscope 20 is inserted while light is connected to endoscope 20 and is being emitted from tip 24, so that the installation of the endoscope can be visually monitored. Further, the placement of tip 24 can be visually confirmed when the field of view shows a portion of the interior of head 12 and the space it surrounds. Also, alternative methods involving insertion of malleable stylets may be variously included, as described above.

As noted earlier, assemblies may be provided with device heads for performing procedures other than manipulation and positioning. For example, FIG. 9 shows a stabilizer assembly 1′ in a disassembled configuration. While usable under other surgical conditions, assembly 1′ is particularly well-suited for use in closed-cavity, such as closed-chest or limited space surgical sites, and provides visualization capability for visualizing sites that would otherwise not be visible under previous practice techniques. Stabilizer 10′ includes a low profile head 12 configured for use in closed-chest or limited space surgical sites, wherein the low profile facilitates insertion of head 12 and occupies only a very narrow space between a target site on the surface of an organ and tissues surrounding the organ. Stabilizer head 12 is adapted to contact the heart or other organ or tissue to provide stabilization thereto during a surgical procedure. Stabilizer head 12 may include a pair of foot members or contact members 12a and 12b as shown in FIG. 9, which may be connected to form a substantial U-shape, and which may be substantially planar, or slightly curved to conform to the shape of the heart or other organ or tissue, or one or more contact members may have a non-conforming curve to establish a contact between only a portion thereof and the beating heart or other organ and tissue. Stabilizer head 12 may be malleable such that the shape of the contact members may be varied depending on the clinical assessment by the surgeon, the design of the remainder of the stabilizer 10′, and/or the design of other instruments to be used to complete an anastomosis.

Stabilizer head 12 may effect stabilization by application of negative pressure to the surface to be stabilized, which draws the surface against the contact members of stabilizer head 12 when the negative pressure is applied. Further, a combination of negative pressure (suction) and physical (mechanical) pressure against the surface to be stabilized is possible. Stabilizer head 12 provides a low profile configuration adapted to engage with and stabilize a portion of the surface of an organ, such as a beating heart for example, with a sufficient strength and rigidity so as not to break contact with the surface of the organ once vacuum has been applied to establish a seal therewith, so that a region of the surface of the organ (the target area) is stabilized relative to the beating motions of the remainder of the beating heart. Contact members 12a, 12b may include seals made of silicone or other biocompatible elastomer, sealed foam, etc, sufficient to facilitate sealing stabilizer head 12 to the surface of the organ under negative pressure.

In order to maintain negative pressure when endoscope 20 has been inserted, and distal tip extends slightly into the working space between contact members 12a and 12b to provide visualization of the working space relative to the contact members, stabilizer head comprises a viewing window structure 12v that extend slightly between contact members 12a, 12b. Viewing window is transparent, at least on the distal face thereof. Viewing window 12v extends the lumen of elongated member 13, through the proximal end portion of stabilizer head 12, to a location between the contact members 12a, 12b as shown. Viewing window 12 forms a vacuum tight seal with the remainder of stabilizer head 12 and elongated member 13, so that endoscope tip 24 can be extended distally of the proximal portion of stabilizer head 12, without losing vacuum, to provide the desired field of view.

Elongated member 13 may further be configured to be transformed from a flexible conformation to a rigid conformation, such as by interlocking links or and/or ball joint members as known in the art. The remaining features of device 10 and endoscope 20 are the same as those described above with regard to assembly 1 in FIG. 1 and function in the same manner. The same variations in the assembly 1 described above are also possible with assembly 1′.

FIG. 10 shows another stabilizer 10″ that may be used in assembly with an endoscope 20, such as endoscope described above in FIG. 1. While usable under other surgical conditions, stabilizer 10″ is particularly well-suited for use in closed-cavity, such as closed-chest or limited space surgical sites, and provides visualization capability for visualizing sites that would otherwise not be visible under previous practice techniques. Stabilizer 10″ includes a low profile head 12 configured for use in closed-chest or limited space surgical sites, wherein the low profile facilitates insertion of head 12 and occupies only a very narrow space between a target site on the surface of an organ and tissues surrounding the organ. Stabilizer head 12 is adapted to contact the heart or other organ or tissue to provide stabilization thereto during a surgical procedure. Stabilizer head 12 may include a pair of foot members or contact members 12a′ and 12b′ as shown in FIG. 10, which may be connected to form a substantial U-shape, and which may be substantially planar, or slightly curved to conform to the shape of the heart or other organ or tissue, or one or more contact members may have a non-conforming curve to establish a contact between only a portion thereof and the beating heart or other organ and tissue. Stabilizer head 12 may be malleable such that the shape of the contact members may be varied depending on the clinical assessment by the surgeon, the design of the remainder of the stabilizer 10″, and/or the design of other instruments to be used to complete an anastomosis.

Stabilizer head 12 may effect stabilization by application of mechanical force against the surface to be stabilized. Stabilizer head 12 provides a low profile configuration adapted to engage with and stabilize a portion of the surface of an organ, such as a beating heart for example, with a sufficient strength and rigidity to stabilize a region of the surface of the organ (the target area) relative to the beating motions of the remainder of the beating heart.

Stabilizer 10″ further includes an elongated member 13 that may be the same as any of the constructions described above for elongate member 13, and which includes a lumen that receives endoscope 20 therethrough. Since this embodiment does not apply suction to perform the stabilization, suction port 19 is not required at the proximal end portion of device 10″.

Further, since this embodiment does not apply vacuum, window 12v is not necessary. Instead, an opening 12o is provided on the distal face of the proximal end portion of stabilizer head 12. Opening 12o opens to an extension of lumen 16 that passes through the proximal end portion of stabilizer head 12 and allows tip 24 to be passed therethrough to be positioned for viewing the working space between contact members 12a′, 12b′ relative to contact members 12a′, 12b′.

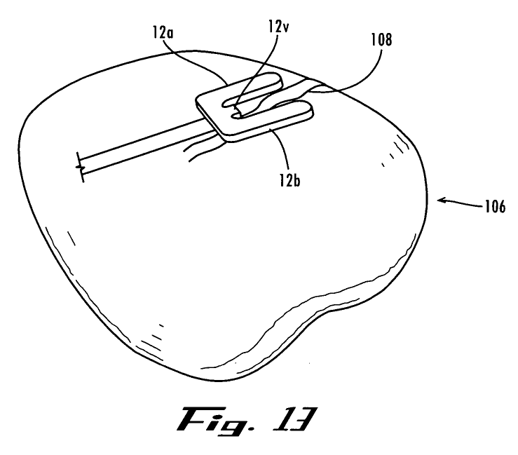

Alternatively, window 12v or opening 12o may be established superiorly of the stabilizer head 12 by passing elongated member 13 over the top of the proximal end portion of stabilizer head 12, as shown in FIGS. 11 and 12, respectively. These alternative configurations may provided better angulation of the field of view.

Alternative stabilizer heads 12 may be utilized to offer a choice of a different design or configuration, or even principle of operation. For example, other known contact members/stabilizer heads could be used or adapted to be used by those of ordinary skill in the art, to include a window 12v or opening 12o as necessary for proper placement of endoscope tip 24. The contact members of stabilizer head 12 may have frictional surfaces on the underside thereof to more securely engage the tissue that they contact. The tips or ends of the contact members may be bent upward in the forms of “ski tips” to prevent edge effects (e.g., stress concentration, cutting, chafing, etc.) against the tissue which might otherwise be caused by straight tips. The contact members may also be modified to include apertures, openings or attachments to facilitate connection with sutures or other devices used to achieve the stabilization and/or anastomosis. Examples of alternative contact members/stabilizer heads that may also be modified for use with the present stabilizer can be found, for example, in U.S. Pat. Nos. 6,036,641; 6,283,912; 5,894,843; 5,727,569; 5,836,311; 5,865,730; or in U.S. Patent Application Publication No. 2002/0099268. U.S. Pat. Nos. 6,036,641; 6,283,912; 5,894,843; 5,727,569; 5,836,311; 5,865,730; and U.S. Patent Application Publication No. 2002/0099268 are each hereby incorporated herein, in their entireties, by reference thereto.

One method of using assembly 1′ to perform stabilization of a target area of a beating heart to perform an anastomosis procedure on a coronary artery of a beating heart during a closed chest procedure is now described. Endoscope 20 is inserted into elongated member 13 of device 10′, through hemostatic valve 17 and advanced until distal tip 24 is in position adjacent window 12v to provide a field of view to allow the user to view where head 12 is being placed. For example, endoscope 20 may be advanced until the operator/surgeon visually confirms tip 24 in position adjacent to and within window extension 12v. The endoscope 20 is connected to a light source (and, optionally to a camera and/or monitor) and device 10 is connected to a suction source, although suction is not yet applied to the device.

Assembly 1′ is next inserted though a port, small thoracotomy, or other small incision in a patient and is advanced toward the target surgical site. Head 12 may be compacted, such as by folding, rolling or otherwise compressing it for easier insertion through the incision in the patient. Alternatively, head 12 may be sufficiently small in an operative configuration so as not to require compression to pass it through the incision. Light may be supplied through endoscope, even prior to passing head 12 through the incision. In its undeformed, operative configuration, head 12 will not obstruct visualization through tip 24 of endoscope 20 and so the advancement of the assembly 1′ may be visualized to assist in directing the assembly to the target location.

Once in the target location, visualization of the field of view 33 over the area that head 12 is currently placed in can confirm whether or not the location is a desirable one for placement of head 12. Another major benefit is that the endoscope can be left in place in the assembly to provide visualization of a surgical procedure (e.g., distal anastomosis) performed while stabilizing the target surgical area. Thus, this arrangement may eliminate the need to insert a rigid endoscope that is currently required to be inserted through another port (other than the one that a current stabilizer is inserted through) to provide visualization of the surgical procedure. Alternatively, after placing a positioner or stabilizer, the endoscope may be removed from the positioner or stabilizer and (or another flexible endoscope as described herein may be) inserted through another port to provide a better viewing angle to visualize the surgical procedure.

In this example, stabilizer head 12 is desired to be placed in an orientation where contact members 12a and 12b straddle the coronary artery to be operated on, as illustrated in FIG. 13. Once the operator/surgeon confirms that the contact members 12a, 12b are positioned properly and do not overlie or intersect the coronary artery, but rather straddle the coronary artery 108, suction is applied through suction port 19 and head 12 forms a seal with the surface of the heart, thereby engaging it under negative pressure. Elongated member 13 may then be fixed or locked in its current configuration. One known example of providing this capability is to provide interlocking links to surround the flexible tube that provides the vacuum proof conduit. A tension member extending the length of such links, when tensioned, locks the interlocking links into the current configuration. Elongated member 13 may then be fixed at its proximal end portion outside of the patient to maintain the current orientation of the stabilizer head 12 to stabilize the area around and at the location of the heart surface contacted, thereby enable further anastomosis procedures to be conducted on a relatively stable site. For example, a clamp, or other mechanical fixation means may be applied to the proximal end portion of elongated member 13 to fix it relative to a stationary object outside the patient to prevent axial, as well as rotational movements thereof. Upon completion of the procedure, elongated member is released from fixation and returned to its flexible configuration. Vacuum is then released from device 10 and head 12 releases its engagement with the surface of the heart. Assembly 1′ can then be withdrawn from the patient. Application of light through tip 24 may be maintained if desired, even during removal of the assembly, to provide visual feedback to the surgeon of the removal process of assembly, and to ensure that the removal process proceeds safely and without incident.

Alternative methods to that described may be practiced with assembly 1′. For example, head 12 may be inserted through the incision prior to insertion of endoscope 20 into device 10. In such case, endoscope 20 may be inserted immediately after passing head 12 through the incision and ensuring that head 12 is in its operable (noncompressed) configuration. Alternatively, head 12 may be advanced part way along the route to the target surgical site prior to inserting endoscope 20. In either case, endoscope 20 is inserted while light is connected to endoscope 20 and is being emitted from tip 24, so that the installation of the endoscope can be visually monitored. Further, the placement of tip 24 can be visually confirmed when the field of view shows a portion of the interior of head 12 and the space it surrounds. Also, alternative methods involving insertion of malleable stylets may be variously included, as described above. Further, methods of using the assembly with a mechanical stabilizer head 12 and opening 12o may include the above described variations.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. An assembly for carrying out a procedure on an organ or tissue in a closed or restricted space surgical site, said assembly comprising:
a head configured and dimensioned to be delivered to a target location on the organ or tissue through an opening in a patient and having a contact surface configured to contact a surface of the organ or tissue at the target location;
an elongated member extending proximally from said head and having sufficient length so that a proximal end portion of said elongated member extends out of the opening in the patient when said contact surface of said head contacts the organ or tissue at the target location, said elongated member having a lumen extending longitudinally therethrough; and
an endoscope dimensioned to be received in said lumen and having an elongated shaft of sufficient length such that when a distal tip of said endoscope extends into said head, and said contact surface contacts the organ or tissue at the target location, a proximal end portion of said elongated shaft extends out of the opening in the patient.

2. The assembly of claim 1, wherein said head comprises a suction member, said suction member having an opening therethrough to extend said lumen into said suction head and to receive said distal tip of said endoscope, and wherein said elongated member connects said suction head with a vacuum source via said lumen.

3. The assembly of claim 2, wherein a proximal end portion of said elongated member includes a hemostatic valve and a suction port, wherein said endoscope is introducible through said hemostatic valve, and the vacuum source is connectable to said lumen though said suction port.

4. The assembly of claim 3, wherein said endoscope is insertable into said lumen and removable therefrom while maintaining suction to said suction member through said lumen.

5. The assembly of claim 1, wherein said lumen functions as a suction conduit even when said endoscope is inserted therethrough to the extent where said distal tip extends into said head.

6. The assembly of claim 1, wherein said elongated member is flexible.

7. The assembly of claim 6, wherein said elongated member is torsionally rigid.

8. The assembly of claim 6, wherein said elongated member is adapted to convert from a flexible configuration to a rigid configuration.

9. The assembly of claim 1, wherein said elongated shaft is flexible.

10. The assembly of claim 1, further comprising a malleable stylet extending coaxially with said elongated shaft.

11. The assembly of claim 10, wherein said malleable stylet is incorporated into said elongated shaft.

12. The assembly of claim 1, wherein said endoscope comprises a fiber optic endoscope.

13. The assembly of claim 1, wherein said endoscope comprises at least one charge coupled device in a distal end portion thereof.

14. The assembly of claim 1, wherein said distal tip is angled with respect to a normal to a longitudinal axis of the elongated shaft when said elongated shaft is in a straight configuration.

15. The assembly of claim 14, wherein said distal tip is angled at an angle of about 30 to about 45 degrees to the normal to the longitudinal axis.

16. The assembly of claim 1, wherein said distal tip is configured to provide a field of view that is angled with respect to the normal to a longitudinal axis of said elongated shaft to provide a downward looking field of view.

17. The assembly of claim 2, wherein said head is a manipulator head and said suction member comprises a suction cup configured to attach to the surface of the organ or tissue under negative pressure with sufficient force so that when said manipulator head is moved, said organ or head moves with said manipulator head, as said manipulator head maintains a seal with the tissue or organ.

19. The assembly of claim 2, wherein said head is a stabilizer head, and said suction member comprises a pair of contact members configured to attach to the surface of the organ or tissue under suction.

20. An organ manipulator assembly for manipulating an organ in a closed-cavity or reduced access surgical site, said assembly comprising:
a suction head having an interior space surrounded by a contact member configured to seal with a surface of the organ when said contact member is contacted to the surface and negative pressure is applied to said interior space;
an elongated member extending proximally from said suction head and having sufficient length so that a proximal end portion of said elongated member extends out of an opening in a patient through which said suction head is inserted and advanced to contact the organ at a target location;
a lumen extending longitudinally through said elongated member;
an opening through said suction head connecting said lumen with said interior space; and
an endoscope dimensioned to be received in said lumen, while still allowing vacuum flow through said lumen, inserted through said lumen so that a distal tip of said endoscope extends into said interior space.

21. The assembly of claim 20, wherein a proximal end portion of said elongated member includes a hemostatic valve and a suction port, wherein said endoscope is introducible through said hemostatic valve, and the vacuum source is connectable to said lumen though said suction port.

22. A method of carrying out a procedure on an organ or tissue in a closed or restricted space surgical site, said method comprising:
providing an assembly having a head and an elongated member with an endoscope received within a lumen of the elongated member such that a distal tip of the endoscope is positioned to visualize placement of said head;
inserting said head through an opening in a patient;
advancing said head to a region of the organ or tissue that includes a target location on which a procedure is to be performed;
visualizing a location of said head, relative to the target location, through said endoscope; and
performing the procedure at the target location with said head after visually confirming that placement of said head, relative to the target location, is acceptable.

23. The method of claim 22, further comprising bending said assembly to a desired conformation prior to said inserting, wherein said assembly includes a malleable stylet running longitudinally therealong that maintains the conformation of said assembly after said bending.

24. The method of claim 22, further comprising applying vacuum through said lumen to attach said head to the target location after said visually confirming that placement of said head, relative to the target location, is acceptable.

25. The method of claim 22, wherein the target location is not visible from the opening in the patient.

26. The method of claim 22, wherein the target location is not accessible along a straight line pathway from the opening in the patient.

27. The method of claim 24, further comprising removing said endoscope from said lumen while maintaining vacuum to said head sufficient to maintain attachment of said head to said organ or tissue.

28. The method of claim 22, wherein the procedure comprises moving the head to reposition the organ or tissue.

29. The method of claim 22, wherein the procedure comprises stabilizing the target location.

30. The method of claim 28, wherein the procedure comprises repositioning a heart.

31. The method of claim 29, wherein the target location is on a beating heart.
